(19) United States
(12) Patent Application Publication
(10) Pub. No.: US 2024/0410896 A1
(43) Pub. Date: Dec. 12, 2024

(54) METHODS AND SYSTEMS OF DETECTING PROTEIN-PROTEIN INTERACTION AND PROBING PROTEIN STRUCTURES

(71) Applicant: The Hong Kong University of Science and Technology, Hong Kong (CN)

(72) Inventors: Weichuan YU, Hong Kong (CN); Ning LI, Hong Kong (CN); Chen ZHOU, Hong Kong (CN); Shuaijian DAI, Hong Kong (CN)

(21) Appl. No.: 18/733,847

(22) Filed: Jun. 5, 2024

Related U.S. Application Data
(60) Provisional application No. 63/506,369, filed on Jun. 6, 2023.

(51) Int. Cl. G01N 33/68; H01J 49/00

(52) U.S. Cl. CPC G01N 33/6845; G01N 33/6851; H01J 49/0068; G01N 2458/15

(57) ABSTRACT

This system presents a comprehensive approach for detecting protein-protein interactions and exploring protein structures. It encompasses various components, including a receptacle for receiving cross-linked precursor peptides, a mass spectrometer for generating MS1 and MS2 spectra, and modules for precursor mass refinement, MS2 spectrum scoring, protein score database construction, and feedback mechanism processing. Through the integration of these elements, the system facilitates the identification of peptide interactions and protein structures, leveraging a cross-linking dataset obtained from proteins cross-linked with cleavable or non-cleavable crosslinking reagents.

### CROSS-REFERENCE TO RELATED APPLICATIONS

The present application claims priority from U.S. provisional patent application Ser. No. 63/506,369 filed Jun. 6, 2023, and the disclosure of which is incorporated herein by reference in its entirety.

### FIELD OF THE INVENTION

The present invention generally relates to proteomics. More specifically the present invention relates to cross-linking mass spectrometry.

### BACKGROUND OF THE INVENTION

Mass spectrometry (MS) has emerged as a pivotal tool for rapidly identifying the primary structure of proteins, revolutionizing the study of biological and pathological phenomena. Chemical cross-linking coupled with mass spectrometry (XL-MS) stands out as a significant application, facilitating the elucidation of proteins' three-dimensional structure and shedding light on protein-protein interactions (PPIs). Given the critical role of understanding protein structure and PPIs in unraveling intricate cellular mechanisms, XL-MS has become indispensable in this field.

In XL-MS experiment, a vital component is the cross-linker, a chemical reagent added to the protein solution. This cross-linker contains two functional groups capable of connecting specific side chains of amino acids, creating covalent bonds that establish spatial constraints within proteins or between interacting proteins. Cross-linkers are categorized as non-cleavable or cleavable. While non-cleavable cross-linkers remain intact during the dissociation process in tandem mass spectrometry (MS2), cleavable cross-linkers feature delicately designed breakable bonds within the linker. Fragmentation of cleavable cross-linkers during dissociation produces reporter ions in the spectrum, reducing computational complexity. Essentially, both types of cross-linkers yield equivalent information about protein structure and PPIs, differing only in the additional signature reporter ions generated by cleavable cross-linkers.

However, both cleavable and non-cleavable XL-MS experiments encounter a significant challenge: the uneven fragmentation efficiency of cross-linked peptide pairs during collision-induced dissociation (CID). This imbalanced fragmentation often leads to incomplete or entirely absent fragmentation of one of the cross-linked peptides, rendering many MS2 spectra unidentifiable and compromising sensitivity.

Various strategies have been proposed to address this issue of imbalanced fragmentation. These strategies include leveraging electron-transfer dissociation (ETD) MS2 spectra, stepped higher-energy collision dissociation (stepped-HCD) MS2 spectra, or collision-induced dissociation MS/MS/MS (MS3) spectra. While these approaches capitalize on hardware features, their application development remains limited, with achieved sensitivity typically ranging from 20% to 50%.

Therefore, the present invention addresses this need and presents a method for identifying cleavable and non-cleavable cross-linking peptides with high sensitivity and accuracy.

### SUMMARY OF THE INVENTION

It is an objective of the present invention to provide a system or method to solve the aforementioned technical problems.

In accordance with a first aspect of the present invention, a system for detecting protein-protein interaction and probing protein structures is provided. Particularly, the system includes the following components: a receptacle for receiving a cross-linked precursor peptide produced by digestion of a protein cross-linked with either cleavable or non-cleavable crosslinker; a mass spectrometer for producing precursor ions and generating MS1 spectrum so as to determine the mass spectrum of the ions with their charge states and masses; and processing at least one precursor ion to undergo a fragmentation treatment to generate an interested MS2 spectrum; a precursor mass refinement module for analyzing an elution profile generated by the mass spectrometer and extracting and merging MS1 spectra obtained before and after the interested MS2 spectrum in the elution profile to form a complete isotope cluster of the precursor ion; a MS2 spectrum scoring module for matching the interested MS2 spectrum with the complete isotope cluster utilizing a scoring function S(x) and detecting and storing top p hits of the MS2 spectrum and their scores, wherein the MS2 spectrum scoring module further retrieves the top a peptide and β peptide hit of the MS2 spectrum as significant peptides and generates a scoring histogram; a protein score database constructed based on the scoring histogram and significant peptides for providing global information to connect peptides to proteins, comprising protein sequences in a biological system; a feedback mechanism processor for retrieving the stored top p hits in the protein score database and identifying at least one peptide candidate in the protein score database, wherein a peptide candidate having the largest protein score among the at least one peptide candidate is identified as a matching result for the interested MS2 spectrum; identifying the peptide interaction or protein structure from the matching result; and a cross-linking dataset, comprising cleavable cross-linker data or non-cleavable cross-linker data, both obtained from proteins in a biological system cross-linked by a cleavable or non-cleavable crosslinking reagent.

In accordance with one embodiment of the present invention, the system may further include a local alignment module configured to provide a series of compensation matches while there is no signature ion available on the interested MS2 spectrum.

In accordance with another embodiment of the present invention, the precursor mass refinement module selects and calibrates the complete isotope cluster with a theoretical isotope cluster to find a monoisotopic mass using a Pearson correlation coefficient with a formula of:

$${γ}_{xy} = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}} ;$$

wherein x and y stand for the theoretical isotope cluster and the complete isotope cluster, respectively.

In accordance with one embodiment of the present invention, the scoring is conducted with the following counting formula:

$$\{ \begin{matrix}C ( x ) = \frac{100}{k} \cdot \frac{1 - {( \frac{100 - k}{100 + k} )}^{x}}{1 + {( \frac{100 - k}{100 + k} )}^{x}} \\ PS = {\sum }_{x ∈ \vec{P}} C ( x )\end{matrix} ;$$

wherein k is the link site(s) number in an individual protein and P is a protein vector, and the protein score PS is calculated by summing the counting result.

In accordance with one embodiment of the present invention, the proteins cross-linked by the cleavable or non-cleavable crosslinking reagent are further filtered, enriched, and digested to produce cross-linked peptides.

In accordance with one embodiment of the present invention, the biological system includes cells, tissues, blood, serum, sputum.

In accordance with one embodiment of the present invention, the precursor mass refinement module has a default correlation cutoff value set as 0.9 to determine the complete isotope cluster.

In accordance with one embodiment of the present invention, the feedback mechanism processor utilizes a XlinkX scoring function as the default for the cleavable cross-linking data:

$${S}_{XlinkX} = 1 - {\sum }_{i = 0}^{n - 1} \frac{{e}^{- xf} ( {xf}^{i} )}{i !} ;$$

wherein

$$x = \frac{4}{111} {M}_{prec} \cdot ϵ ;$$

ϵ is the MS2 tolerance (ppm); and f is equal to

$$\frac{α}{α + β} {f}_{total}$$

when β peptide score is calculated, where α and β denote α and β peptide mass and fₜₒₜₐₗ is the total number of fragmented ions in the MS2 spectrum.

In accordance with one embodiment of the present invention, wherein the feedback mechanism processor utilizes a Xcorr scoring function as the default for the non-cleavable cross-linking data:

$${S}_{Xcorr} = \vec{e} \cdot \vec{t} ;$$

wherein \vec{e} is the vectorization form of the input MS2 spectrum and \vec{t} is the vectorized theoretical peptide sequence generated from the fragmented peptide N terminal ions (b ions) and C terminal ions (y ions).

In accordance with a second aspect of the present invention, a method of detecting protein-protein interaction and probing protein structures utilizing the aforementioned system is provided. Specifically, the method includes the following steps:
obtaining a cross-linked precursor peptide produced by digestion of a protein cross-linked with a cleavable or non-cleavable crosslinker as a sample;
subjecting the sample to the mass spectrometry to produce precursor ions and generate MS1 spectra used to determine the charge states and masses of precursor ions;
processing at least one precursor ion of the precursor ions to undergo a fragmentation treatment to generate an interested MS2 spectrum;
extracting and merging MS1 spectra obtained before and after the interested MS2 spectrum in an elution profile to form a complete isotope cluster of the precursor ion utilizing the precursor mass refinement module;
inputting the MS2 spectrum to the MS2 spectrum scoring module for matching the interested MS2 spectrum with the complete isotope cluster;
detecting and storing top p of α and β peptide hits of the MS2 spectrum and their scores, wherein the MS2 spectrum scoring module further retrieves the top p of a peptide and β peptide hits of the MS2 spectrum as significant peptides and generates a scoring histogram;
constructing the protein score database using the significant peptides and the scoring histogram;
retrieving the stored top p of a peptide and β peptide hits in the protein score database so as to identify at least one peptide candidate denoted with sequence from the protein score database, wherein the peptide candidate having the largest protein score among the at least one peptide candidate is identified as a matching result for the input MS2 spectrum; and
re-matching the sequence by using the protein score and outputting a cross-linked peptide-spectrum match for identifying the peptide interaction or protein structure from the matching result.

In accordance with one embodiment of the present invention, the MS2 spectrum is de-charged if the MS2 spectrum matches with the cleavable cross-linker data.

In accordance with another embodiment of the present invention, the method further includes adopting a target decoy database to control the output quality.

In accordance with one embodiment of the present invention, the target decoy database is constructed by reversing the protein sequences.

In accordance with one embodiment of the present invention, the sample is further filtered, enriched, and digested to produce cross-linked peptides.

In accordance with one embodiment of the present invention, in the step of retrieving, further catching top 20 hits for further justification after matching.

### BRIEF DESCRIPTION OF THE DRAWINGS

Embodiments of the invention are described in more details hereinafter with reference to the drawings, in which:

FIGS. 1A-1D depicts a workflow of the precursor mass refinement module of one embodiment of the present invention, in which FIG. 1A shows that the nearby MS1 spectra are merged to generate a complete isotope cluster, FIG. 1B displays that the isotope cluster of the merged MS1 is compared with that of the single (original) MS1 and the fusion result has a better shape and is more similar to the theoretical isotope cluster, FIG. 1C depicts that the theoretical isotope cluster is compared with each possible position in the MS1 spectrum, and the Pearson coefficient is used to measure the similarity, and FIG. 1D depicts the system of one embodiment of the present invention;

FIGS. 2A-2B depict a workflow of the method of detecting protein-protein interaction and probing protein structures according to one embodiment of the present invention, in which FIG. 2A demonstrates an operation workflow of the method and FIG. 2B illustrates the protein feedback mechanism in one embodiment;

FIGS. 3A-3B depict the performance of the system of one embodiment of the present invention, in which FIG. 3A depicts the comparisons between the system ECL 3.0 and current analysis tool on non-cleavable XL-MS data and FIG. 3B depicts the comparisons between the system ECL 3.0 and current analysis tool on cleavable XL-MS data;

FIGS. 4A-4B depict the comparisons among different systems using synthetic datasets where the true positive results are denoted by a dark grey bar and false positives are denoted by a light grey bar, in which FIG. 4A shows the cross-link spectrum matches (CSMs) and unique cross-link results for DSBU datasets, and FIG. 4B plots the CSMs and unique cross-link results for DSSO datasets;

FIGS. 5A-5B depict the sensitivity and precision comparisons among different systems using simulated datasets, in which FIG. 5A displays the sensitivity evaluation and FIG. 5B shows the precision evaluation; and

FIGS. 6A-6C depict a protein structure validation using the system of the present invention in one embodiment, in which FIG. 6A shows the identified cross-links mapped onto the X-ray diffraction structure of the GroEL complex (PDB ID: 1KP8), FIG. 6B demonstrates a 90-degree rotation of the structure model shown in FIG. 6A, and FIG. 6C displays the histogram of Cα-Cα distance of all cross-links within the GroEL complex with a distance cutoff of 30 Å.

### DETAILED DESCRIPTION

In the following description, systems, and/or methods of detecting protein-protein interaction and probing protein structures and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Addressing the challenge of identifying poorly fragmented peptides in cross-linking mass spectrometry (XL-MS), the present invention introduces a system and/or method leveraging additional information for peptide identification. While designing scoring functions aids in finding correct peptides, it incompletely resolves the issue. The proposed system/method overcomes this limitation by incorporating additional information. Conventionally, database searching relies solely on peptide sequence data from the FASTA file to match the spectrum, overlooking the correlation between protein and peptide sequences. This oversight results in underutilization of protein database information. For instance, when two similar peptides yield identical or very similar scores, but one peptide has numerous confidently identified “sibling” peptides from other MS2 spectra while the other lacks such identifications, the former should be prioritized as the correct match. Building on this concept, a protein feedback pipeline exploits this relationship, yielding substantially heightened sensitivity compared to existing techniques.

As used herein, the terms “MS/MS” and “tandem mass spectrometry” are interchangeable and refer to mass spectrometric analysis with two stages.

In XL-MS, the process typically involves obtaining both MS1 and MS2 spectra. In the first step, the mass spectrometer scans all ions within a certain mass range (typically the entire mass range of interest) and records their abundance (intensity) at each mass-to-charge ratio (m/z). This produces the MS1 spectrum, which provides information about the ions present in the sample and their relative abundances. The MS1 spectrum helps identify precursor ions of interest for further analysis.

Once precursor ions of interest are identified from the MS1 spectrum, they are subjected to fragmentation by collision-induced dissociation (CID) or other fragmentation methods. This fragmentation generates a spectrum of fragment ions, known as the MS2 spectrum. The MS2 spectrum provides information about the structure and composition of the precursor ion, including the amino acid sequence in the case of peptide analysis. In XL-MS, the MS2 spectrum helps identify cross-linked peptides and elucidate their structure.

As used herein, the term “MS1 spectrum”, also known as the precursor ion spectrum, is generated in mass spectrometry during the first stage of analysis. It records the intensity of ions corresponding to intact precursor molecules (ions) within a sample, providing information about their mass-to-charge ratios (m/z) and abundance. The MS1 spectrum aids in identifying and quantifying molecules present in the sample by measuring their mass and abundance before fragmentation occurs.

As used herein, the term “MS2 spectrum”, also known as the product ion spectrum, is generated in mass spectrometry during the second stage of analysis. It results from the fragmentation of precursor ions, typically achieved by collision-induced dissociation (CID) or other fragmentation methods. The MS2 spectrum displays the resulting fragment ions produced from the precursor ions, providing information about the structural composition of molecules within the sample. Analysis of the MS2 spectrum helps elucidate the primary structure of molecules and identify specific molecular fragments, aiding in compound identification and structural characterization.

As used herein, the term “elution profile” represents the distribution of ions detected by the mass spectrometer as a function of elution time. This profile provides information about the retention times of different peptides, which is crucial for identifying and quantifying peptides in the subsequent mass spectrometry analysis steps. In XL-MS, the elution profile of an MS2 spectrum refers to the collection of MS1 spectra obtained before and after the specific MS2 spectrum of interest. These MS1 spectra are merged to form a complete isotope cluster, which is used to determine the precursor mass and facilitate peptide identification. Therefore, the elution profile plays a vital role in precursor mass refinement, a crucial step in XL-MS data analysis.

In accordance with a first aspect of the present invention, a system for detecting protein-protein interaction and probing protein structures is provided.

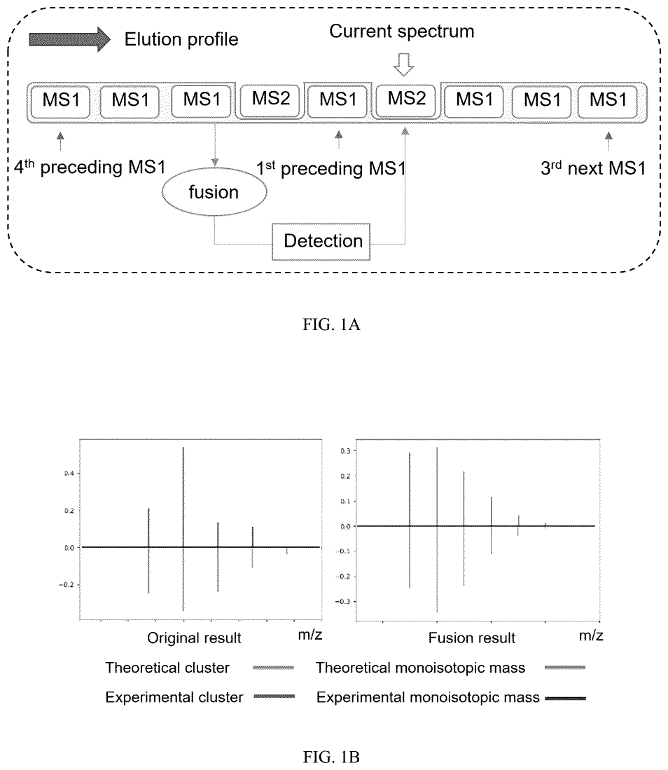

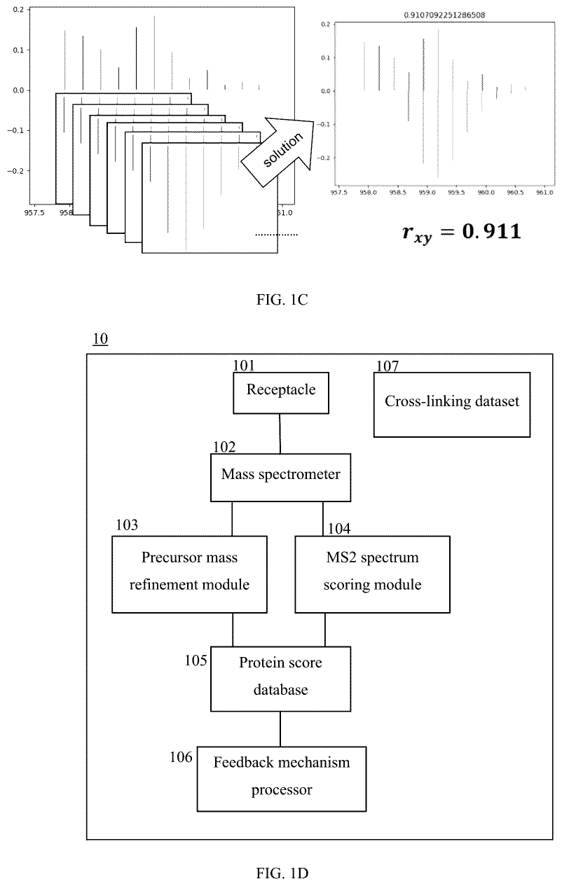

Referring to FIG. 1D, the system 10 includes several integrated components designed to facilitate comprehensive analysis. It includes a receptacle 101 for receiving cross-linked precursor peptides obtained through the digestion of proteins cross-linked with a cleavable or non-cleavable crosslinker. These precursor peptides serve as the basis for subsequent analysis. A mass spectrometer 102 is employed to produce precursor ions and generate MS1 spectrum, providing crucial information about the mass spectrum of ions along with their charge states and masses. Additionally, the mass spectrometer 102 processes at least one precursor ion to undergo a fragmentation treatment, resulting in the generation of an interested MS2 spectrum, which is pivotal for further analysis.

A precursor mass refinement module 103 analyzes the elution profile generated by the mass spectrometer 102, meticulously extracting and merging MS1 spectra obtained before and after the interested MS2 spectrum. This process aims to form a complete isotope cluster of the precursor ion, enhancing the accuracy of subsequent analyses. A pivotal component, the MS2 spectrum scoring module 104, matches the interested MS2 spectrum with the complete isotope cluster using a scoring function S(x). It detects and stores top p hits of the MS2 spectrum along with their scores, and further retrieves the top a peptide and B peptide hit of the MS2 spectrum as significant peptides. Additionally, it generates a scoring histogram, providing valuable insights into peptide interactions.

Moreover, the system 10 includes a protein score database 105 constructed based on the scoring histogram and significant peptides. This database offers global information to connect peptides to proteins, comprising protein sequences in a biological system. A feedback mechanism processor 106 retrieves the stored top p hits in the protein score database, identifying at least one peptide candidate. Among these candidates, the one with the largest protein score is identified as a matching result for the interested MS2 spectrum. This process enables the identification of peptide interactions or protein structures from the matching result.

Furthermore, the system 10 incorporates a cross-linking dataset 107 including cleavable or non-cleavable cross-linker data. These data are obtained from proteins in a biological system cross-linked by a cleavable or non-cleavable crosslinking reagent, providing essential information for comprehensive analysis.

In some embodiments, the system 10 may further include specific modules for precursor mass refinement and scoring, ensuring the accuracy and efficiency of the analysis process. Additionally, the system 10 has a default correlation cutoff value and scoring functions further enhance the system's performance, enabling robust analysis of protein-protein interactions and protein structures. For instance, the system may include a local alignment module (not shown) configured to provide a series of compensation matches while there is no signature ion available on the interested MS2 spectrum.

The precursor mass refinement module 103 selects and calibrates the complete isotope cluster with a theoretical isotope cluster to find a monoisotopic mass using a Pearson correlation coefficient. The scoring process is conducted with a counting formula. The proteins cross-linked by the cleavable or non-cleavable crosslinking reagent are further filtered, enriched, and digested to produce cross-linked peptides. The biological system may include cells, tissues, blood, serum, or sputum.

The feedback mechanism processor 106 utilizes a XlinkX scoring function as the default for cleavable cross-linking data, while a Xcorr scoring function is used as the default for non-cleavable cross-linking data.

In accordance with a second aspect of the present invention, a method of detecting protein-protein interaction and probing protein structures, utilizing the aforementioned system, is provided. The method involves several intricately coordinated steps aimed at comprehensive analysis.

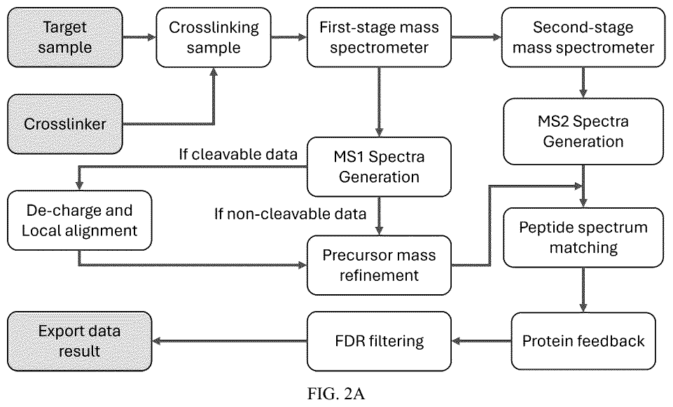

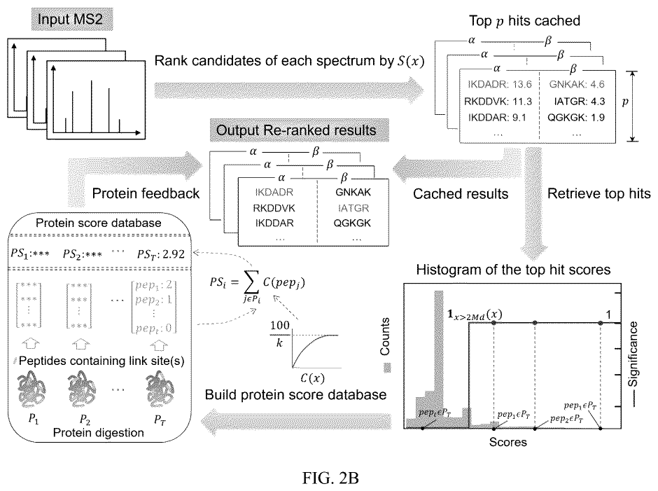

Referring to FIG. 2A, initially, a cross-linked precursor peptide is obtained through the digestion of a protein cross-linked with a cleavable or non-cleavable crosslinker, serving as the sample for analysis. This sample is subjected to mass spectrometry, wherein precursor ions are produced, and MS1 spectra are generated to determine the charge states and masses of precursor ions. Subsequently, at least one precursor ion undergoes a fragmentation treatment to generate an interested MS2 spectrum, which is pivotal for further analysis.

The method further involves the extraction and merging of MS1 spectra obtained before and after the interested MS2 spectrum in an elution profile, facilitated by the precursor mass refinement module. This process aims to form a complete isotope cluster of the precursor ion, enhancing the accuracy of subsequent analyses. The interested MS2 spectrum is then inputted into the MS2 spectrum scoring module for matching with the complete isotope cluster. Top p of a peptide and B peptide hits of the MS2 spectrum, along with their scores, are detected and stored, with the module retrieving the top p of a peptide and β peptide hits as significant peptides and generating a scoring histogram.

Moreover, the method includes constructing a protein score database using the significant peptides and the scoring histogram, which provides vital information for subsequent analysis. The stored top p of α and β hits in the protein score database are retrieved to identify at least one peptide candidate denoted with sequence from the protein score database. The peptide candidate with the largest protein score among the candidates is identified as a matching result for the input MS2 spectrum. Subsequently, the sequence is rematched using the protein score, outputting a cross-linked peptide-spectrum match for identifying the peptide interaction or protein structure from the matching result.

In specific embodiments, the method involves de-charging the MS2 spectrum if it matches with the cleavable cross-linker data, and adopting a target decoy database to control the output quality, wherein the target decoy database is constructed by reversing the protein sequences. Additionally, the sample may be further filtered, enriched, and digested to produce cross-linked peptides. Moreover, in the step of retrieving, the method may further involve catching the top 20 hits for further justification after matching.

Precursor mass refinement is a critical phase in the presented XL-MS system, ensuring precise precursor mass information for accurate peptide identification. In this process, the system approximates the theoretical isotope distribution and compares it with the experimental MS1 spectrum to determine the correct precursor mass. Illustrated in FIG. 1A, this module's workflow involves extracting and merging nearby MS1 spectra of the current MS2 spectrum to generate a consolidated spectrum. Utilizing four preceding and three subsequent MS1 spectra, this step also limits the scan event range. Comparing this fused isotope cluster with the theoretical one, as depicted in FIG. 1B, reveals closer similarity, indicating improvement. Subsequently, as shown in FIG. 1C, the system evaluates each possible position of the theoretical isotope cluster within the new MS1 spectrum, computing the Pearson correlation coefficient using Eq. 1. The resulting coefficient indicates the best-fit precursor mass. If no coefficient exceeds the predefined threshold (typically 0.9), the system retains the precursor mass provided in the RAW file.

$${γ}_{xy} = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}} ; \qquad ( 1 )$$

where x and y stand for the theoretical and experimental isotope clusters, respectively.

In the subsequent step, the scoring function S(x) is employed to directly match the query spectra. This function retains the top p peptide candidates alongside their respective scores for further assessment. In a particular embodiment, users are offered three primary alternative scoring functions for selection in cleavable cross-linking searches: the default simplified XlinkX scoring function outlined in Eq. 2, a normalized MeroX scoring function delineated in Eq. 3, and an Xcorr scoring function (SEQUEST) as detailed in Eq. 4. For non-cleavable cross-linking searches, the Xcorr scoring function (SEQUEST) is made available.

$${S}_{XlinkX} = 1 - {\sum }_{i = 0}^{n - 1} \frac{{e}^{- xf} ( {xf}^{i} )}{i !} ; \qquad ( 2 )$$

in which

$$x = \frac{4}{111} {M}_{prec} \cdot ϵ ;$$

ϵ is the MS2 tolerance (ppm); and f is equal to

$$\frac{α}{α + β} {f}_{total}$$

when β peptide score is calculated, where α and β denote α and β peptide mass and fₜₒₜₐₗ is the total number of fragmented ions in the MS2 spectrum.

$${S}_{MeroX} = - 50 log [ 0.1 {e}^{- \frac{k}{i}} + 0.05 {e}^{- \frac{20 h}{d}} + 0.1 {e}^{- \frac{k}{6}} + \frac{0.3}{z} {20}^{- {F}_{int}^{2}} ] + 50 log [ 0.25 + \frac{0.3}{z} ] , \qquad ( 3 )$$

here k is the number of identified abundant ions (≥10% intensity); i is the number of abundant peaks in the MS2 spectrum; h is the number of identified ions; d is the total fragmented peaks in the MS2 spectrum; and Fᵢₙₜ is the ratio of the intensity of identified ions to that of total ions in the MS2 spectrum.

$${S}_{Xcorr} = \vec{e} \cdot \vec{t} ; \qquad ( 4 )$$

in which \vec{e} is the vectorization form of the experimental MS2 spectrum and \vec{t} is the vectorized theoretical peptide sequence generated from the b/y ions.

Following the scoring of peptides, the algorithm proceeds to retrieve the top hits of each spectrum and constructs a histogram of their scores in the subsequent step. It is imperative for a well-designed scoring function to exhibit the characteristic that higher scores correspond to more confident peptide matches. To achieve this, an indicator function is utilized to categorize peptides based on their scores in a binary manner. Empirically, the significant domain is identified as twice the median or the 98th percentile of the scores. Subsequently, all significant peptides are employed in constructing a protein score database.

A concept of a protein score database is introduced as follows: Each protein contained within the FASTA file undergoes in silico digestion into peptides. These peptides, which contain the link site(s), are then utilized to construct a vector corresponding to the individual protein. The length of the vector matches the number of peptides generated. Upon identification of a specific peptide of a protein in the significant area of the histogram m times, the corresponding position in the vector is assigned the value m. Upon completion of the enumeration of all proteins in the FASTA file, the counting function C(x) from Eq. 5 is applied to transfer the counts and aggregate all counting results from the protein vector to derive the final protein score, denoted as PS. Differing from peptide databases utilized to provide potential peptide sequences for identifying experimental spectra, the protein score database furnishes comprehensive information to establish connections between peptides and proteins. This database is established using the theoretical protein sequence and the scoring function.

$$\{ \begin{matrix}C ( x ) = \frac{100}{k} \cdot \frac{1 - {( \frac{100 - k}{100 + k} )}^{x}}{1 + {( \frac{100 - k}{100 + k} )}^{x}} \\ PS = {\sum }_{x ∈ \vec{P}} C ( x )\end{matrix} , \qquad ( 5 )$$

here k is the link site(s) number in the individual protein and \vec{P} is the protein vector. The protein score PS is calculated by summing the counting result.

The subsequent phase involves the feedback process. The algorithm revisits the stored top hits and cross-references the peptide candidates in the protein score database. Within the pool of peptide candidates, the one exhibiting the highest protein score is deemed the peptide match for that spectrum.

Finally, the target decoy false discovery rate (FDR) strategy is implemented to regulate false positives, employing Equation 6.

$$FDR := \frac{\# TT - \# DD}{\# TD} , \qquad ( 6 )$$

here, T represents target hits and D represents decoy hits.

In summary, the feedback mechanism has a general workflow. The input MS2 spectra are first matched with peptide sequences by the scoring function S(x). The algorithm stores the top p hits for each spectrum during the matching process. We then retrieve the first α and β peptide hits of all the spectra and plot the histogram of the score distribution in a further step. Adopting an indicator function (2M d is twice the median number), a protein score database is built using the filtered (significant) peptides. Each protein in the database is first digested into peptides, and only the peptides containing a link site(s) are used to construct a vector for that protein. Next, the counting function C(x) is utilized to compute the protein score. In the last step, the stored top p peptides are retrieved for identifying the largest protein score among these candidates so as to finish the matching process and output the results.

Referring to FIG. 2B, protein T can be digested into t distinct peptide sequences. Its vector length is t. In the histogram, sequence pep1 is identified twice, and sequence pep2 is identified once in the significant area. Therefore, the vector of protein T is initialized as shown in the protein score database. After enumerating all proteins in the FASTA file, the counting function C(x) is operated to transfer the counts and sum up all the counting results from the protein vector to obtain the final protein score PS. If protein T has 20 link sites in its sequence, the final score can be calculated as 2.92. Different from peptide databases that are used to provide potential peptide sequences to identify experimental spectra, the protein score database is used to provide global information to connect peptides to proteins and is established by the theoretical protein sequence and the scoring function. The last step is the feedback procedure. The algorithm returns to the stored top hits and looks up the peptide candidates in the protein score database. Among the peptide candidates, the one having the largest protein score is regarded as the peptide match for that spectrum. Testing on a proteome-wide dataset, it is found that the protein feedback pipeline can universally improve the performance of the scoring functions (SXlinkX, SMeroX, and SXcorr) by 29.7%.

### EXAMPLES

### Example 1. Evaluation Using Synthetic Dataset

MS cleavable datasets are downloaded from the synthetic cross-linked peptide library (PXD014337). DSSO and DSBU are used to cross-link 95 synthesized peptides. PD 2.4 MS Annika, PD 2.4 XlinkX 60-day free version, MaxLynx (MaxQuant 2.0.3.0), and MeroX 2.0.1.4, as well as the system of the present invention (ECL-PF), are used to analyze these datasets. Local alignment and precursor mass refinement modules are enabled in ECL-PF.

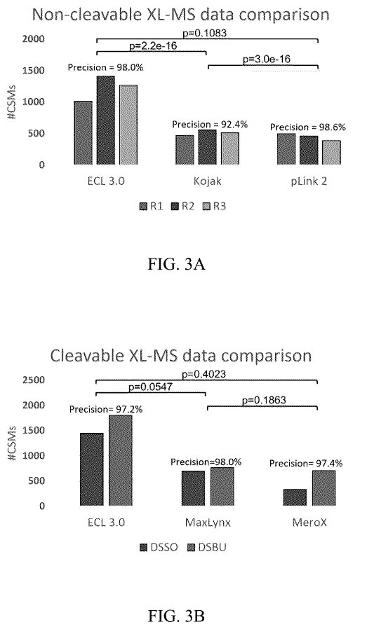

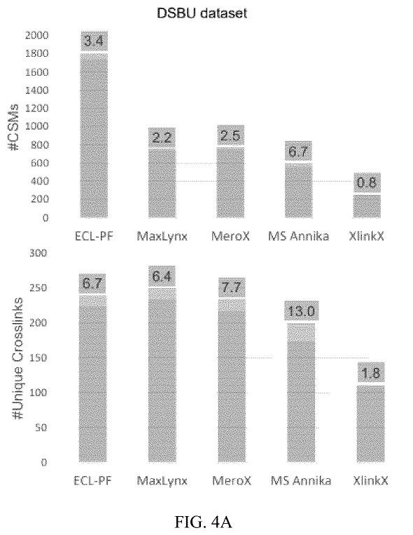

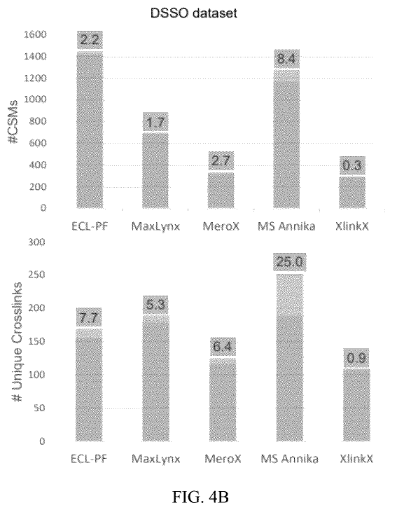

The results for the number of cross-link spectrum matches (CSMs) and unique cross-links at FDR=5% are shown in FIGS. 4A-4B. Among the CSMs results, ECL-PF always obtains the highest true positive numbers. On average, ECL-PF identifies 2.3 times as many CSMs as other software on the DSBU dataset (FIG. 4A) and 3.0 times as many CSMs as the others on the DSSO dataset (FIG. 4B). For the unique cross-links, due to the limited number of synthesized peptides in the sample, ECL-PF does not outperform all the others but is in the same range as the best one. From the calculated FDR, it is observed that MS Annika suffers from a high proportion of false positives—on the DSSO dataset, one quarter of the unique cross-links are wrong. Meanwhile, it is observed that XlinkX obtains the worst results for all datasets. Thus, MS Annika and XlinkX are removed from the comparisons hereafter.

### Example 2. Evaluation Using Hybrid Simulated Dataset

Due to the unique characteristics of XL-MS, a test is designed to generate a simulated dataset based on a real experimental one. The signal peaks of the α and β peptides are separated from the noise peaks in each identified spectrum and the α peaks are combined from one spectrum with the β peaks from another spectrum to produce a new spectrum. In this way, spectra with quantity of N can generate N² simulated spectra with ground truth. Additionally, their noise peaks can be added into the new spectrum so as to have more realistic spectra. Here, the synthetic cross-linked peptide library is utilized as the source to generate the simulated dataset and tune the noise ratio to make the spectra more complicated.

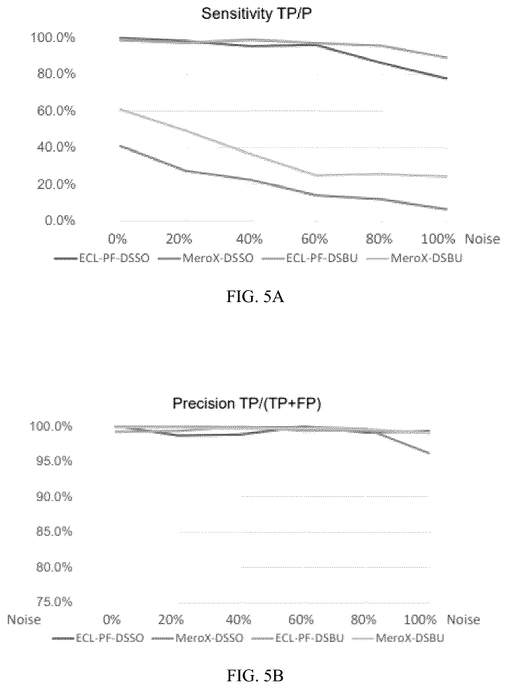

MaxLynx cannot function with simulated datasets because its input only allows RAW files. Therefore, in this section, ECL-PF is compared only with MeroX. To fairly compare them, the overlapping spectra identified by both software are utilized to generate our simulated dataset. To test their robustness, the noise peaks are also added into the spectra. In total, 25,221 spectra for the DSSO cross-linker and 84,741 spectra for the DSBU cross-linker are generated. Among them, the noise peaks extracted from the original spectra are added. The noise proportion varies from 0% to 100%, where 100% means that all noise peaks from the original spectrum are added into the new spectrum. FIG. 5A and FIG. 5B show the sensitivity and precision comparison between ECL-PF and MeroX. ECL-PF identifies almost all the cross-linked spectra when there is no noise, whereas MeroX identifies half of them on average. As the noise proportion increasing, both of their sensitivities drop. ECL-PF can still identify more than 80% of the spectra in the worst case, but MeroX can only identify 20%. They both satisfy our FDR at the 5% setting. Simulated results show that ECL-PF can not only achieve better result but is also more robust to noise than MeroX.

### Example 3. Evaluation Using Real Dataset

Four real datasets are used to evaluate the performance of ECL-PF, MaxLynx, and MeroX. The crosslinker varies from DSSO and DSBU to DSBSO. FDR at 1% is set for the four sets of comparisons. The precursor mass refinement module and local alignment module are enabled for ECL-PF. Table 1 shows the result of the comparisons. For all the tested data, ECL-PF outperforms the others by a large margin in terms of CSMs number and unique cross-links. On average, ECL-PF identifies 91.6% more CSMs and 52.6% more unique cross-links than the other methods in these real experimental datasets.

TABLE 1
Real dataset comparison among Maxlynx, MeroX and ECL-PF

| | | MaxLynx | | MeroX | | ECL-PF | |
|---|---|---|---|---|---|---|---|
| Data | | CSMs | Cross-links | CSMs | Cross-links | CSMs | Cross-links |
| In vitro tests | PXD011861 | 4627 | 794 | 2511 | 672 | 5369 | 847 |
| | PXD016963 | 1957 | 204 | 806 | 108 | 2665 | 333 |
| | PXD020014 | 1888 | 101 | 613 | 78 | 4566 | 209 |
| In vivo tests | PXD012546 | 31754 | 8211 | 28826 | 9422 | 48075 | 10260 |

| | | | | | | |
|---|---|---|---|---|---|---|
| Ratio | 66.8% | 70.9% | 37.6% | 60.2% | 100% | 100% |

### Example 4. Structure Validation

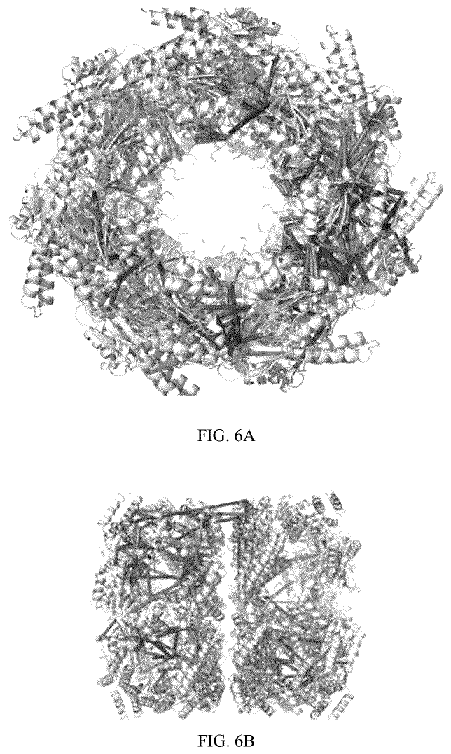

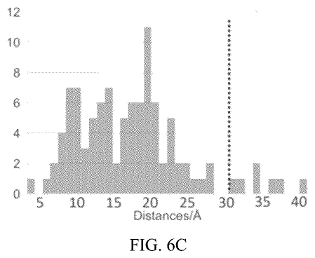

E. coli GroEL complex results from ECL-PF are filtered from the real experimental dataset. The X-ray diffraction structure of the GroEL (PDB ID: 1KP8) is downloaded and the cross-linked results obtained form the system of the present invention are mapped onto the protein structure. In total, 101 pairs of Lysine-Lysine sites are identified from ECL-PF, where 93.1% of the Cα-Cα distances satisfy the cross-linker's constraint. The protein structure and histogram of the distances are shown in FIGS. 6A-6C. Particularly, FIG. 6A shows the identified cross-links (black lines) mapped onto the X-ray diffraction structure of the GroEL complex (PDF ID: 1KP8), the 90-degree rotation of this structure model is further illustrated in FIG. 6B. FIG. 6C displays the histogram of Cα-Cα distance of all cross-links within the GroEL complex, where 30 Å is set as the distance cutoff shown in the dotted line. Overall, this structure study shows the solidity of ECL-PF from the experimental point of view.

In summary, the present invention has revealed that utilizing protein-peptide association during the identification process can significantly improve sensitivity. Furthermore, it is demonstrated that major scoring functions in XL-MS can all benefit greatly from this association. By implementing a protein feedback mechanism, the sensitivity of non-cleavable and cleavable cross-linking data analysis can be improved by three-fold (on a synthetic data set with controlled false positive proportion).

Therefore, the present systems and methods offer potential applications across diverse fields such as drug discovery, biotechnology, agriculture, and the food industry. For drug discovery, the system can serve as a platform to identify novel drug targets and to screen and optimize drug candidates. Moreover, it can facilitate the identification of protein-protein interactions crucial for understanding disease mechanisms and developing effective therapies. In the biotechnology sector, the system can optimize the production of protein-based therapeutics, including monoclonal antibodies, and aid in the development of biosensors and other protein-based diagnostic tools. Additionally, the system can contribute to agricultural advancements by studying protein-protein interactions in plants and developing disease-resistant crop varieties. Furthermore, it can enhance food industry practices by elucidating the structure and interactions of proteins in food, thereby improving the quality and safety of food products.

Furthermore, the system can be used to elucidate the three-dimensional structure of proteins by identifying intramolecular and intermolecular cross-linking peptide bonds. This information is crucial for understanding protein folding, conformational changes, and interactions with other molecules.

XL-MS is valuable for studying protein-protein interactions (PPIs) within biological systems. By identifying cross-linked peptides, the system can reveal the proximity and spatial arrangement of proteins in complexes, providing insights into cellular signaling pathways, molecular recognition events, and disease mechanisms.

Additionally, XL-MS can aid in the discovery of biomarkers for various diseases and conditions. By analyzing protein complexes and interactions in biological samples, the system can identify specific cross-linked peptides associated with disease states, potentially leading to the development of diagnostic tools and therapeutic targets.

XL-MS has broad applications in biomedical research, including studies of protein dynamics, post-translational modifications, and cellular signaling pathways. The system enables researchers to investigate complex biological processes at the molecular level, facilitating discoveries in areas such as cancer biology, neurodegenerative diseases, and infectious diseases.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A system for detecting protein-protein interaction and probing protein structures, comprising:
a receptacle for receiving a cross-linked precursor peptide produced by digestion of a protein cross-linked with a cleavable or a non-cleavable crosslinker;
a mass spectrometer for producing precursor ions and generating MS1 spectrum so as to determine the mass spectrum of the ions with their charge states and masses; and processing at least one precursor ion to undergo a fragmentation treatment to generate an interested MS2 spectrum;
a precursor mass refinement module for analyzing an elution profile generated by the mass spectrometer and extracting and merging MS1 spectra obtained before and after the interested MS2 spectrum in the elution profile to form a complete isotope cluster of the precursor ion;
a MS2 spectrum scoring module for matching the interested MS2 spectrum with the complete isotope cluster utilizing a scoring function S(x) and detecting and storing top p hits of the MS2 spectrum and their scores, wherein the MS2 spectrum scoring module further retrieves the top a peptide and β peptide hit of the MS2 spectrum as significant peptides and generates a scoring histogram;
a protein score database constructed based on the scoring histogram and significant peptides for providing global information to connect peptides to proteins, comprising protein sequences in a biological system;
a feedback mechanism processor for retrieving the stored top p hits in the protein score database and identifying at least one peptide candidate in the protein score database, wherein a peptide candidate having the largest protein score among the at least one peptide candidate is identified as a matching result for the interested MS2 spectrum; identifying the peptide interaction or protein structure from the matching result; and
a cross-linking dataset, comprising cleavable cross-linker data or non-cleavable cross-linker data, both obtained from proteins in a biological system cross-linked by a cleavable or non-cleavable crosslinking reagent.

2. The system of claim 1, further comprising a local alignment module configured to provide a series of compensation matches while there is no signature ion available on the interested cleavable crosslinking MS2 spectrum.

3. The system of claim 1, wherein the precursor mass refinement module selects and calibrates the complete isotope cluster with a theoretical isotope cluster to find a monoisotopic mass using a Pearson correlation coefficient with a formula of:
$${γ}_{xy} = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}} ;$$
wherein x and y stand for the theoretical isotope cluster and the complete isotope cluster, respectively.

4. The system of claim 1, wherein the scoring is conducted with the following counting formula:
$$\{ \begin{matrix}C ( x ) = \frac{100}{k} \cdot \frac{1 - {( \frac{100 - k}{100 + k} )}^{x}}{1 + {( \frac{100 - k}{100 + k} )}^{x}} \\ PS = {\sum }_{x ∈ \vec{P}} C ( x )\end{matrix} ;$$
wherein k is the link site(s) number in an individual protein and \vec{P} is a protein vector, and the protein score PS is calculated by summing the counting result.

5. The system of claim 1, wherein the proteins cross-linked by the cleavable or non-cleavable crosslinking reagent are further filtered, enriched, and digested to produce cross-linked peptides.

6. The system of claim 1, wherein the biological system comprises cells, tissues, blood, serum, sputum.

7. The system of claim 2, wherein the precursor mass refinement module has a default correlation cutoff value set as 0.9 to determine the complete isotope cluster.

8. The system of claim 1, wherein the feedback mechanism processor utilizes a XlinkX scoring function as the default for the cleavable cross-linking data:
$${S}_{XlinkX} = 1 - {\sum }_{i = 0}^{n - 1} \frac{{e}^{- xf} ( {xf}^{i} )}{i !} ;$$
wherein
$$x = \frac{4}{111} {M}_{prec} \cdot ϵ ;$$
ϵ is the MS2 tolerance (ppm); and f is equal to
$$\frac{α}{α + β} {f}_{total}$$
when β peptide score is calculated, where α and β denote α and β peptide mass and fₜₒₜₐₗ is the total number of fragmented ions in the MS2 spectrum.

9. The system of claim 1, wherein the feedback mechanism processor utilizes a Xcorr scoring function as the default for the non-cleavable cross-linking data:
$${S}_{Xcorr} = \vec{e} \cdot \vec{t} ;$$
wherein \vec{e} is the vectorization form of the input MS2 spectrum and t is the vectorized theoretical peptide sequence generated from fragmented peptide N terminal ions (b ions) and C terminal ions (y ions).

10. A method of detecting protein-protein interaction and probing protein structures utilizing the system of claim 1, comprising:
obtaining a cross-linked precursor peptide produced by digestion of a protein cross-linked with a cleavable or a non-cleavable crosslinker as a sample;
subjecting the sample to the mass spectrometry to produce precursor ions and generate MS1 spectra used to determine the charge states and masses of precursor ions;
processing at least one precursor ion of the precursor ions to undergo a fragmentation treatment to generate an interested MS2 spectrum;
extracting and merging MS1 spectra obtained before and after the interested MS2 spectrum in an elution profile to form a complete isotope cluster of the precursor ion utilizing the precursor mass refinement module;
inputting the MS2 spectrum to the MS2 spectrum scoring module for matching the interested MS2 spectrum with the complete isotope cluster;
detecting and storing top p of a peptide and β peptide hits of the MS2 spectrum and their scores, wherein the MS2 spectrum scoring module further retrieves the top p of α and β hits of the MS2 spectrum as significant peptides and generates a scoring histogram;
constructing the protein score database using the significant peptides and the scoring histogram
retrieving the stored top p of α and β hits in the protein score database so as to identify at least one peptide candidate denoted with sequence from the protein score database, wherein the peptide candidate having the largest protein score among the at least one peptide candidate is identified as a matching result for the input MS2 spectrum; and
re-matching the sequence by using the protein score and outputting a cross-linked peptide-spectrum match for identifying the peptide interaction or protein structure from the matching result.

11. The method of claim 10, wherein the MS2 spectrum is de-charged if the MS2 spectrum matches with the cleavable cross-linker data.

12. The method of claim 10, further comprising adopting a target decoy database to control the output quality.

13. The method of claim 12, wherein the target decoy database is constructed by reversing the protein sequences.

14. The method of claim 10, wherein the sample is further filtered, enriched, and digested to produce cross-linked peptides.

15. The method of claim 10, in the step of retrieving, further catching top 20 hits for further justification after matching.
